(19) United States
(12) Patent Application Publication
(10) Pub. No.: US 2019/0240131 A1
(43) Pub. Date: Aug. 8, 2019

(54) AQUEOUS SURFACTANT COMPOSITIONS

(71) Applicant: BASF SE, Ludwigshafen am Rhein (DE)

(72) Inventors: Claudia Brunn, Düsseldorf-Holthausen (DE); Ansgar Behler, Düsseldorf-Holthausen (DE)

(21) Appl. No.: 16/317,132

(22) PCT Filed: May 11, 2017
(86) PCT No.: PCT/EP2017/061285

§ 371 Date: Jan. 11, 2019

(30) Foreign Application Priority Data
Jul. 19, 2016 (EP) 16180121.2

(51) Int. Cl. A61K 8/46; A61K 8/898; A61K 8/892; A61K 8/891; A61Q 11/00; A61Q 9/02; A61Q 19/10; A61Q 5/02; C11D 11/00; C11D 1/37; C11D 3/37; C11D 3/04; C11D 3/34

(52) U.S. Cl. CPC A61K 8/466; C11D 1/28; A61K 8/892; A61K 8/891; A61Q 11/00; A61Q 9/02; A61Q 19/10; A61Q 5/02; C11D 11/0023; C11D 1/37; C11D 3/373; C11D 3/3742; C11D 3/046; C11D 3/349; A61K 2800/596; A61K 8/898

(57) ABSTRACT

Aqueous surfactant compositions comprising one or more alpha-sulfo fatty acid disalt (A) of general formula (I),

R¹CH(SO₃M¹)COOM² (I),
in which the radical R¹ is a linear or branched alkyl or alkenyl radical having 6 to 18 carbon atoms and the radicals M¹ and M², independently of one another, are selected from the group comprising H, Li, Na, K, Ca/2, Mg/2, ammonium and alkanolamine,
one or more silicone compound (B) selected from amodimethicones (B1), dimethicones (B2), dimethiconols (B3) and cyclomethicones (B4), and water.

The compositions have good foaming ability, a pleasant sensory feel to the foam, good skin compatibility, and are suitable for cosmetic products, detergents, and cleaners.

### FIELD OF THE INVENTION

The present invention relates to aqueous surfactant compositions with a content of alpha-sulfo fatty acid disalts and specific silicone compounds.

### PRIOR ART

Anionic surfactants are some of the most widespread interface-active compounds and, apart from being used in detergents and cleaners, are also used for diverse purposes in the field of cosmetics. Customary anionic surfactants as are used in particular in cosmetics are the salts of alkyl ether sulfates (alkyl polyether sulfates, fatty alcohol polyglycol ether sulfates, in short also ether sulfates). They are characterized by a strong foaming ability, high cleaning power, low sensitivity to hardness and grease and are used widely for producing cosmetic products such as, for example, hair shampoos, foam or shower baths, but also in hand dishwashing detergents.

For many current applications, apart from a good interface-active effect, further requirements are placed on anionic surfactants. A high dermatological compatibility is required in particular in cosmetics. In addition, good foaming ability and a pleasant sensory feel to the foam are generally desired. Furthermore, there is a need for anionic surfactants which can be produced at least partially from biogenic sources and specifically also renewable raw materials.

### DESCRIPTION OF THE INVENTION

The object of the present invention was to provide aqueous surfactant compositions which are characterized by the properties specified below:
good foaming ability.
pleasant sensory feel to the foam.
good skin compatibility.

The invention firstly provides aqueous surfactant compositions comprising
one or more alpha-sulfo fatty acid disalts (A) of general formula (I),

R¹CH(SO₃M¹)COOM² (I),

in which the radical R¹ is a linear or branched alkyl or alkenyl radical having 6 to 18 carbon atoms and the radicals M¹ and M² independently of one another are selected from the group comprising H, Li, Na, K, Ca/2, Mg/2, ammonium and alkanolamines,
one or more silicone compounds (B) selected from the group formed by amodimethicones (B1), dimethicones (B2), dimethiconols (B3) and cyclomethicones (B4), and
water,
   where the following provisos apply:
if the aqueous surfactant compositions comprises one or more ester sulfonates (E) of general formula (V),

R²CH(SO₃M⁷)COOR³ (V)

in which the radical R² is a linear or branched alkyl or alkenyl radical having 6 to 18 carbon atoms and the radical R³ is a linear or branched alkyl or alkenyl radical having 1 to 20 carbon atoms, where the radical R³ can logically be an alkenyl radical or be branched only above 3 carbon atoms, and the radical M⁷ is selected from the group comprising Li, Na, K, Ca/2, Mg/2, ammonium and alkanolamines, it is the case that the compounds (A) based on the totality of the compounds (A) and (E) must be present to an extent of 50% by weight or more—and in particular to an extent of 90% by weight or more.
The amodimethicones (B1) have the general formula (IIa)

HO—Si(CH₃)₂—O—[Si(CH₃)₂—O]ₙ—[Si(OH)((CH₂)₃NH(CH₂)₂NH₂)—O]ₘ—Si(CH₃)₂—OH (IIa)

in which the index n is an integer between 10 and 100 000 and the index m is an integer between 2 and 20 000.
The dimethicones (B2) have the general formula (IIb)

(CH₃)₃Si—O—[Si(CH₃)₂—O]ₓ—Si(CH₃)₃ (IIb)

in which the index x is an integer between 10 and 100 000.
The dimethiconols (B3) have the general formula (IIc)

OH—Si(CH₃)₂—O—[Si(CH₃)₂—O]_{y}Si(CH₃)₂—OH (IIc)

in which the index y is an integer between 10 and 100 000.
The cyclomethicones (B4) have the general formula (IId)

[Si(CH₃)₂O]_{z} (IId)

in which the index z is an integer between 3 and 6.

The aqueous surfactant compositions according to the invention are characterized by the following advantageous properties:
Good foaming ability and pleasant sensory feel to the foam. In this regard, it may be noted that particularly in the field of cosmetics, foaming ability can be understood to mean different aspects, for example it being possible to use any of foam volume, foam stability, foam elasticity, water content of the foam as well as optical features of the foam such as, for example, the pore size, for the purpose of assessing the foam. The compositions according to the invention have a large foam volume during the initial foaming. In practice, the initial foaming takes place within a relatively short period (from a few seconds to one minute). Typically, during initial foaming, a shower gel or a shampoo is spread and caused to foam by rubbing between hands, skin and/or hair. In the laboratory, the foaming behavior of an aqueous surfactant solution can be assessed e.g. by agitating the solution within a comparatively short time period by means of stirring, shaking, pumping, bubbling through a gas stream or in another way. Subjective assessment of the foam sensory feel can be made in volunteer tests. For this purpose, aspects such as, for example, creaminess, elasticity, moldability of the foam may be assessed.
Good skin and mucosa compatibility. These can be detected by in vitro methods known to those skilled in the art (e.g. RBC or HET-CAM) and also in volunteer tests (e.g. patch test).
Outstanding care performance on skin and hair. This can be assessed, for example in volunteer tests by reference to subjective skin feel (smoothness, dryness etc.) or haptics and feel of the treated hair. Mechanical measurement methods, such as combability of the hair, for example, can also be used.
Good storage stability. This is then the case if the aqueous compositions do not experience any visible (e.g. cloudiness, discoloration, phase separation) or measurable (e.g. pH, viscosity, active substance content) changes over a period of several weeks.
Good applicability and processibility. The compositions can be dissolved rapidly and without supply of heat on introduction into water.
Good clear solubility and transparency. The aqueous surfactant compositions do not have a tendency to precipitation or cloudiness.
Sufficiently high viscosity, which is understood in the context of the present invention to mean a value of 1000 mPas or higher (measured with a Brookfield RV laboratory rheometer at 23° C., 12 rpm, spindle set RV 02 to 07 (spindle choice depending on viscosity range)). As is known, “mPas” means millipascal seconds.
Good cleaning performance. The aqueous surfactant compositions are suitable for removing and emulsifying soiling, especially fat- or oil-containing soiling, from solid or textile surfaces.

### The Compounds (A)

The compounds (A), which are referred to within the context of the present invention as alpha-sulfo fatty acid disalts, are obligatory for the aqueous surfactant compositions according to the invention. They have the formula (I) specified above

R¹CH(SO₃M¹)COOM² (I),

in which the radical R¹ is a linear or branched alkyl or alkenyl radical having 6 to 18 carbon atoms and the radicals M¹ and M² independently of one another are selected from the group comprising H, Li, Na, K, Ca/2, Mg/2, ammonium and alkanolamines. In this connection, particularly preferred alkanolamines are monoethanolamine, diethanolamine, triethanolamine and monoisopropanolamine.

In one embodiment, the proviso applies that the fraction of the compounds (A) in the aqueous surfactant compositions in which the radical R¹ is an alkenyl radical based on the total amount of the compounds (A) is 3% by weight or less.

In a preferred embodiment, the radical R¹ in the formula (I) is a saturated, linear alkyl radical having 10 to 16 carbon atoms, where with regard to the compounds (A) it is the case that the fraction of the compounds (A) in which the radical R¹ is a decyl and/or a dodecyl radical based on the total amount of the compounds (A)—is 70% by weight or more and preferably 90% by weight or more.

The radicals M¹ and M² in formula (I) are preferably selected from the group comprising H (hydrogen) and Na (sodium).

The compounds (A) can be prepared by all methods known appropriately to those skilled in the art. A particularly preferred method of preparation here is the sulfation of the corresponding carboxylic acids. Here, the corresponding carboxylic acid and in particular the corresponding fatty acids are reacted with gaseous sulfur trioxide, the sulfur trioxide being used preferably in an amount such that the molar ratio of SO₃ to fatty acid is in the range from 1.0:1 to 1.1:1. The crude products obtained in this way, which are acidic sulfation products, are then partially or completely neutralized, preference being given to complete neutralization with aqueous NaOH. If desired, it is also possible to undertake purification steps and/or a bleaching (for adjusting the desired pale color of the products).

In a particularly preferred embodiment, the compounds (A) are used in technical-grade form. This means that the corresponding carboxylic acids, in particular native fatty acid, are sulfated with gaseous sulfur trioxide, as a result of which, following partial or complete neutralization of the resulting acidic sulfation products, a mixture of the compounds (A), (C) and (D) results. By virtue of corresponding adjustments of the reaction parameters (in particular molar ratio of carboxylic acid and sulfur trioxide, and also reaction temperature) it is possible to control the ratio of the compounds (A), (C) and (D). The compounds (C) and (D) are described below in the chapter “Preferred embodiments”.

Within the context of the present invention, preference is given to those technical-grade mixtures of the alpha-sulfo fatty acid disalts which have the following composition:
the content of (A) is in the range from 60 to 100% by weight,
the content of (C) is in the range from 0 to 20% by weight,
the content of (D) is in the range from 0 to 20% by weight,
   with the proviso that the sum of the components (A), (C) and (D) in this mixture is 100% by weight.

### The Compounds (B)

The compounds (B), which are referred to in the context of the present invention as silicone compounds, are obligatory for the aqueous surfactant compositions according to the invention.

The compounds (B) are selected from the group formed by amodimethicones (B1), dimethicones (B2), dimethiconols (B3) and cyclomethicones (B4).

The amodimethicones (B1) have the general formula (IIa)

HO—Si(CH₃)₂—O—[Si(CH₃)₂—0]ₙ—[Si(OH)((CH₂)₃NH(CH₂)₂NH₂)—0]ₘ—Si(CH₃)₂—OH (IIa)

in which the index n is an integer between 10 and 100 000 and the index m is an integer between 2 and 20 000.

The dimethicones (B2) have the general formula (IIb)

(CH₃)₃Si—O—[Si(CH₃)₂—O]ₓ—Si(CH₃)₃ (IIb)

in which the index x is an integer between 10 and 100 000.

The dimethiconols (B3) have the general formula (IIc)

OH—Si(CH₃)₂—O—[Si(CH₃)₂—O]_{y}Si(CH₃)₂—OH (IIc)

in which the index y is an integer between 10 and 100 000.

The cyclomethicones (B4) have the general formula (IId)

[Si(CH₃)₂O]_{z} (IId)

in which the index z is an integer between 3 and 6.

The compounds (B) can be prepared by all of the methods known appropriately to the person skilled in the art. For example, methylchlorosilanes are firstly formed as intermediates by reacting silicon with methyl chloride (Mller-Rochow synthesis). Silanols and cyclosiloxanes are formed by subsequent hydrolysis of the methylchlorosilanes, which may in turn be converted to the desired end products by polycondensation or anionically or cationically initiated ring-opening polymerization.

### PREFERRED EMBODIMENTS

In one embodiment, the aqueous surfactant compositions according to the invention additionally comprise, besides the compounds (A), (B) and water, one or more compounds (C) of general formula (III)

R⁴COOM⁵ (III)

In the formula (111), the radical R⁴ is a linear or branched alkyl or alkenyl radical having 7 to 19 carbon atoms and the radical M⁵ is selected from the group comprising H, Li, Na, K, Ca/2, Mg/2, ammonium and alkanolamines. In this connection, particularly preferred alkanolamines are monoethanolamine, diethanolamine, triethanolamine and monoisopropanolamine.

In one embodiment, the aqueous surfactant compositions according to the invention additionally comprise, besides the compounds (A), (B) and water, one or more inorganic salts of sulfuric acid (D) of general formula (IV)

(M⁶)₂SO₄ (IV)

wherein M⁶ is selected from the group comprising Li, Na, K, Ca/2, Mg/2, ammonium and alkanolamine. In this connection, particularly preferred alkanolamines are monoethanolamine, diethanolamine, triethanolamine and monoisopropanolamine.

In a preferred embodiment, the aqueous surfactant compositions according to the invention comprise the compounds (A), (B), (C) and (D). It is particularly preferable in this case if the radicals M¹ and M² of the compounds (A), the radical M⁵ of the compounds (C) and the radical M⁶ of the compounds (D) are selected from the group comprising H (hydrogen) and Na (sodium).

In one embodiment, the aqueous surfactant compositions according to the invention additionally comprise, besides the compounds (A), (B) and water, one or more compounds (F) of general formula (VI)

R⁶CH₂—CO—CHR⁷(SO₃M⁵) (VI),

in which the radicals R⁶ and R⁷ independently of one another are a linear or branched alkyl radical having 6 to 18 carbon atoms and the radical M⁵ is selected from the group comprising H, Li, Na, K, Ca/2, Mg/2, ammonium and alkanolamines. In this connection, particularly preferred alkanolamines are monoethanolamine, diethanolamine, triethanolamine and monoisopropanolamine.

In the context of the present invention, the compounds (F) are referred to as monosulfoketones.

In a preferred embodiment, the radicals R⁶ and R⁷ in the formula (VI)—independently of one another—are a saturated, linear radical having 10 to 16 carbon atoms, where with regard to the compounds (F) it is the case that the fraction of the compounds (F) in which the radicals R⁶ and R⁷ are a decyl and/or a dodecyl radical based on the total amount of the compounds (F)—is 70% by weight or more and preferably 90% by weight or more. The radical M⁵ in formula (VI) is preferably selected from the group comprising H and Na.

In one embodiment, the aqueous surfactant compositions according to the invention additionally comprise, besides the compounds (A), (B) and water, one or more compounds (G) of general formula (VII)

(SO₃M⁹)R⁸CH—CO—CH)R⁹(SO₃M¹⁰) (VII),

in which the radicals R⁸ and R⁹ independently of one another are a linear or branched alkyl radical having 6 to 18 carbon atoms and the radicals M⁹ and M¹⁰ independently of one another are selected from the group comprising H, Li, Na, K, Ca/2, Mg/2, ammonium and alkanolamines. In this connection, particularly preferred alkanolamines are monoethanolamine, diethanolamine, triethanolamine and monoisopropanolamine.

In the context of the present invention, the compounds (G) are referred to as disulfoketones.

In a preferred embodiment, the radicals R⁸ and R⁹ in the formula (VII)—independently of one another—are a saturated, linear radical having 10 to 16 carbon atoms, where with regard to the compounds (G) it is the case that the fraction of the compounds (G) in which the radicals R⁸ and R⁹ are a decyl and/or a dodecyl radical based on the total amount of the compounds (G)—is 70% by weight or more and preferably 90% by weight or more. The radicals M⁹ and M¹⁰ in formula (VII) are preferably selected from the group comprising H and Na.

The preparation of the compounds (F) and (G) is not subject to any particular restrictions and they can be prepared by all methods known to those skilled in the art.

In one embodiment, the compounds (F) and (G) are prepared by sulfonation of corresponding ketones with gaseous sulfur trioxide, as described in the German published specification DE-A-42,20,580.

In another embodiment, the preparation of the compounds (F) and (G) starts from fatty acids. In this case, the sulfation of liquid fatty acids with gaseous sulfur trioxide is conducted such that, in addition to disalts (A), the compounds (F) and (G) are also formed in this case, which can be accomplished as a result of carrying out the sulfation as follows: the ratio of fatty acid raw materials, which may also be used in the form of mixtures of fatty acids of different chain length, to sulfur trioxide is adjusted so that 1.0 to 1.5 mol and especially 1.0 to 1.25 mol of SO₃ are used per mole of fatty acid(s). The fatty acids are introduced into the reactor at a reservoir temperature in the range of 70 to 100° C. After the sulfation, the resulting liquid sulfation product is maintained and aged at this temperature for 5 to 20 minutes in a temperature-controlled post-reaction coil. Neutralization is then effected with an aqueous base, preferably sodium hydroxide, generally in a pH range of 5 to 10, especially 5 to 7. Subsequently, an acidic bleaching—the pH here is adjusted to a value of 7 or less—may be carried out with hydrogen peroxide.

In one embodiment, the aqueous surfactant compositions according to the invention comprise the compounds (A), (B) and (F). It is particularly preferable in this case if the radicals M¹ and M² of the compounds (A) are selected from the group comprising H (hydrogen) and Na (sodium). The proviso applies in this case that the amount of the compounds (A) must be greater than the amount of the compounds (F).

In one embodiment, the aqueous surfactant compositions according to the invention comprise the compounds (A), (B) and (G). It is particularly preferable in this case if the radicals M¹ and M² of the compounds (A) are selected from the group comprising H and Na. The proviso applies in this case that the amount of the compounds (A) must be greater than the amount of the compounds (G).

In one embodiment, the aqueous surfactant compositions according to the invention comprise the compounds (A), (B), (F) and (G). It is particularly preferable in this case if the radicals M¹ and M² of the compounds (A) are selected from the group comprising H and Na. The proviso applies in this case that the amount of the compounds (A) must be greater than the sum total of the amount of the compounds (F) and (G).

In one embodiment, the aqueous surfactant compositions according to the invention comprise the compounds (A), (B), (C), (D) and (F). It is particularly preferable in this case if the radicals M¹ and M² of the compounds (A), the radical M⁵ of the compounds (C) and the radical M⁶ of the compounds (D) are selected from the group comprising H and Na. The proviso applies in this case that the amount of the compounds (A) must be greater than the amount of the compounds (F).

In one embodiment, the aqueous surfactant compositions according to the invention comprise the compounds (A), (B), (C), (D) and (G). It is particularly preferable in this case if the radicals M¹ and M² of the compounds (A), the radical M⁵ of the compounds (C) and the radical M⁶ of the compounds (D) are selected from the group comprising H and Na. The proviso applies in this case that the amount of the compounds (A) must be greater than the amount of the compounds (G).

In one embodiment, the aqueous surfactant compositions according to the invention comprise the compounds (A), (B), (C), (D), (F) and (G). It is particularly preferable in this case if the radicals M¹ and M² of the compounds (A), the radical M⁵ of the compounds (C) and the radical M⁶ of the compounds (D) are selected from the group comprising H and Na. The proviso applies in this case that the amount of the compounds (A) must be greater than the sum total of the amount of the compounds (F) and (G).

If desired, the aqueous surfactant compositions according to the invention can additionally comprise one or more further surfactants which, in structural terms, do not belong to the aforementioned compounds (A), (B), (D), (E), (F) or (G). These surfactants may be anionic, cationic, nonionic or amphoteric surfactants.

### Use of the Compositions

A further subject matter of the invention is the use of the aforementioned compositions for cosmetic products, and also detergents and cleaners.

With regard to cosmetic products, particular preference is given here especially to those which are present in the form of hair shampoos, shower gels, soaps, syndets, washing pastes, washing lotions, scrub preparations, foam baths, oil baths, shower baths, shaving foams, shaving lotions, shaving creams and dental care products (for example toothpastes, mouthwashes and the like).

With regard to cleaners, of preference here are in particular products with a low pH for cleaning hard surfaces, such as bath and toilet cleaners and the like, and also for cleaning and/or fragrance gels for use in sanitary installations.

1. An aqueous surfactant composition comprising
one or more alpha-sulfa fatty acid disalt (A) of general formula
R¹CH(SO₃M¹)COOM² (I)
in which the radical R¹ is a linear or branched alkyl or alkenyl radical having 6 to 18 carbon atoms and the radicals M¹ and M², independently of one another, are selected from the group consisting of H, Li, Na, K, Ca/2, Mg/2, ammonium, and alkanolamine,
one or more silicone compound (B) selected from the group consisting of amodimethicones (B1), dimethicones (B2), dimethiconols (B3) and cyclomethicones (B4), and
water,
where the following provisos apply:
if the aqueous surfactant compositions comprises one or more ester sultanate (E) of general formula (V),
R²CH(SO₃M⁷)COOR³ (V)
in which the radical R² is a linear or branched alkyl or alkenyl radical having 6 to 18 carbon atoms and the radical R³ is a linear or branched alkyl or alkenyl radical having 1 to 20 carbon atoms, where the radical R³ can be an alkenyl radical or be branched only above 3 carbon atoms, and the radical M⁷ is selected from the group comprising consisting of Li, Na, K, Ca/2, Mg/2, ammonium, and alkanolamine, it is the case that the compound (A), based on the totality of the compounds (A) and (E), must be present to an extent of 50% by weight or more.
the amodimethicones (B1) have a general formula (IIa),
HO—Si(CH₃)₂—O—[Si(CH₃)₂—O]ₙ—[Si(OH)((CH₂)₃NH(CH₂)₂NH₂)—O]ₘ—Si(CH₃)₂—OH (IIa)
in which index n is an integer between 10 and 100 000 and index m is an integer between 2 and 20 000,
the dimethicones (B2) have a general formula (IIb),
(CH₃)₃Si—O—[Si(CH₃)₂—O]ₓ—Si(CH₃)₃ (IIb)
in which index x is an integer between 10 and 100 000,
the dimethiconols (B3) have a general formula (IIc),
OH—Si(CH₃)₂—O—[Si(CH₃)₂—O]_{y}—Si(CH₃)₂—OH (IIc)
in which index y is an integer between 10 and 100 000, and
the cyclomethicones (B4) have a general formula (IId),
[Si(CH₃)₂O], (IId)
in which index z is an integer between 3 and 6.

2. The composition according to claim 1, wherein the radical R¹ in the formula (I) is a saturated, linear alkyl radical having 10 to 16 carbon atoms, where with regard to the compound (A) it is the case that the fraction of the compound (A) in which the radical R¹ is a decyl or a dodecyl radical, based on the total amount of the compounds (A), is 90% by weight or more.

3. The composition according to claim 1, wherein the radicals M¹ and M² are selected from the group consisting of H (hydrogen) and Na (sodium).

4. The composition according to claim 1, wherein the composition additionally comprises one or more compound (C) of general formula (III),
R⁴COOM⁵ (III)
in which the radical R⁴ is a linear or branched alkyl or alkenyl radical having 7 to 19 carbon atoms and the radical M⁵ is selected from the group comprising consisting of H, Li, Na, K, Ca/2, Mg/2, ammonium, and alkanolamine.

5. The composition according to claim 1, wherein the composition additionally comprises one or more inorganic salt of sulfuric acid (D) of general formula (IV),
(M⁶)₂SO₄ (IV)
wherein M⁶ is selected from the group consisting of Li, Na, K, Ca/2, Mg/2, ammonium, and alkanolamine.

6. The composition according to claim 1, wherein the composition additionally one or more monosulfoketone (F) of general formula (VI),
R⁶CH₂—CO—CHR⁷(SO₃M⁸) (VI)
in which the radicals R⁶ and R⁷, independently of one another, are a linear or branched alkyl radical having 6 to 18 carbon atoms and the radical M⁸ is selected from the group consisting of H, Li, Na, K, Ca/2, Mg/2, ammonium, and alkanolamine.

7. The composition according to claim 1, wherein the composition additionally one or more disulfoketone (G) of general formula (VII),
(SO₃M⁹)R⁸CH—CO—CHR⁹(SO₃M¹⁰) (VII)
in which the radicals R⁸ and R⁹, independently of one another, are a linear or branched alkyl radical having 6 to 18 carbon atoms and the radicals M⁹ and M¹⁰, independently of one another, are selected from the group consisting of H, Li, Na, K, Ca/2, Mg/2, ammonium, and alkanolamine.

8. The composition according to claim 1 for use in cosmetic products, detergents, and cleaners.

9. The composition according to claim 1 for use in cosmetic products in the form of hair shampoos, shower gels, soaps, syndets, washing pastes, washing lotions, scrub preparations, foam baths, oil baths, shower baths, shaving foams, shaving lotions, shaving creams, and dental care products.

10. The composition according to claim 1 for use in products having a low pH for cleaning hard surfaces.
